(19) United States
(12) Patent Application Publication
(10) Pub. No.: US 2014/0221854 A1
(43) Pub. Date: Aug. 7, 2014

(54) MEASURING DEVICE, INCLUDING A HEART RATE SENSOR, CONFIGURED TO BE WORN ON THE WRIST OF A USER

(71) Applicant: National Electronics and Watch Company, Aberdeen (HK)

(72) Inventor: Kwong Yuen WAI, Aberdeen (HK)

(73) Assignee: National Electronics and Watch Company, Aberdeen (HK)

(21) Appl. No.: 14/150,699

(22) Filed: Jan. 8, 2014

Related U.S. Application Data
(60) Provisional application No. 61/749,912, filed on Jan. 8, 2013.

(51) Int. Cl. A61B 5/024; A61B 5/00

(52) U.S. Cl. CPC A61B 5/02444; A61B 5/6824
USPC 600/508

(57) ABSTRACT

According to various aspects of the present disclosure, an Optical Heart Rate Sensor is designed to pick up the heart rate pulse when worn on a user's wrist instead of wearing a bulky chest strap, which is not comfortable. An example optical heart rate sensor unit has two light emitting diodes that provide light source projected to skin. An optical detector mounted close to the light source can detect the movement of blood under the skin of the wrist based on light reflected from the skin. The optical detector can detects blood movement by emitting light onto skin and measuring the amount of light absorbed by skin. The optical sensor can be very sensitive to light, electrical static and electromagnetic waves. A special chamber is designed to protect the sensor unit so that the sensor unit can work when user wears the sensor on the arm.

### TECHNICAL FIELD

The present disclosure relates to a measuring device carried by a user during exercise for measuring non-invasively at least one signal from the body. More particularly, the disclosure is directed to an optical heart rate sensor configured to be worn on a user's wrist.

### BACKGROUND

Various portable personal measuring devices for measuring a signal of the user's choice from the body have been designed during the last few years. Devices have been designed for different end users: persons concerned with their health, fitness enthusiasts, goal-oriented athletes, and sports champions.

Signals to be measured include, for example, heart rate and arterial blood pressure. These measurements can be carried out non-invasively, i.e. the measuring sensors are disposed on a person's skin. Hence the use of such measuring sensors is safe and suitable for everyone.

A measuring device designed for measuring heart rate, i.e. a heart rate monitor, for example, is employed to improve physical and mental condition efficiently and safely. The user can employ a heart rate monitor to monitor his heart rate level during exercising, for example, and avoid excessive stress. A heart rate monitor can also be utilized in slimming since it has been scientifically shown that the most efficient way to burn fat stored in the body is to exercise at a given heart rate (about 55 to 65%) of a person's maximum heart rate. The maximum heart rate is calculated e.g. by subtracting the person's age from 220, or the maximum heart rate can also be measured.

In U.S. Pat. No. 4,625,733 to Säynäjäkangas teaches a wireless and continuous heart rate measuring concept employing a transmitter attached to a user's chest for ECG accurate measuring of the user's heart rate and for telemetric transfer of heart rate data to a heart rate receiver attached to the user's wrist by employing magnetic coils in the transfer. The transmitter includes an uncomfortable, bulky chest strap to be worn by the user.

In addition to a receiver, the unit attached to the wrist comprises a control unit and a user interface. The control unit controls and monitors the operation of the measuring device. The necessary heart rate data processing is also carried out in the control unit. The control unit is typically a microprocessor also comprising an ROM memory in which the software of the measuring device is stored. The control unit can also comprise separate memory in which measurement data generated during the use of the device can be stored for further processing. For further processing, the data can be transferred to a separate personal computer.

The user interface of a heart rate monitor comprises selection means for making selections, and display means for displaying data. The selection means are typically push buttons. The number of buttons may vary. A conventional liquid crystal display typically serves as the display means.

The user operates the heart rate monitor by pressing the buttons. The heart rate monitor provides feedback on its display as text, numbers and various symbols.

The basic structure of the user interface in nearly all known heart rate monitors comprises different operating modes. A heart rate monitor usually comprises at least a watch mode and a heart rate measurement mode. In watch mode the heart rate monitor operates as a normal wrist watch. An operating mode may also have sub-operating modes somehow associated with the operating mode. In sub-operating modes, different parameters associated with exercising are displayed to the user. The time of day is a parameter indicating real exercise time. The date can be displayed. An alarm clock type of sub-operating mode is also common.

Different parameters measured for the exercise are displayed in heart rate measurement mode. Examples of sub-operating modes are e.g. exercise time and heart rate, real exercise time and heart rate, effective exercise time and heart rate, energy consumed by the user in the exercise and heart rate. In heart rate measurement mode the user can also be controlled by means of sound signals and symbols displayed on the display. The control may aim at keeping the exercise within effective and safe limits (typically within the range 55 to 85% of a person's maximum heart rate). In this case the user himself typically sets the lower and upper limits for his heart rate. The limits are established on the basis of information obtained in medical studies. During exercise, the measuring device gives an alarm if the heart rate exceeds the upper limit or falls below the lower limit.

The operating modes often also comprise a set mode. The set mode allows the user to set functions controlling and facilitating the exercise, e.g. the lower and upper limits for the heart rate.

The operating modes may also comprise a file mode. This is subject to the device comprising memory for storing data during exercise in the manner described above. In file mode the stored data can be studied and analyzed later.

It may be desirable to provide a heart rate sensor capable of reliably measuring the heart rate of a user without the need for a bulky chest strap. Particularly, it may be desirable to provide a wrist-worn device comprising an optical heart rate sensor module capable of reliably measuring the heart rate of a user.

### SUMMARY

According to various aspects of the present disclosure, an Optical Heart Rate Sensor is designed to pick up the heart rate pulse when worn on a user's wrist instead of wearing a bulky chest strap, which is not comfortable. An example optical heart rate sensor unit has two light emitting diodes that provide light source projected to skin. An optical detector mounted close to the light source can detect the movement of blood under the skin of the wrist based on light reflected from the skin. The optical detector can detects blood movement by emitting light onto skin and measuring the amount of light absorbed by skin.

The optical sensor can be very sensitive to light, electrical static and electromagnetic waves. A special chamber is designed to protect the sensor unit so that the sensor unit can work when user wears the sensor on the arm. The special chamber can protect the sensitive sensor unit from extraneous ambient light, electrical static, and electromagnetic waves. The special chamber can also assist in detecting the weak signal of reflected light indicating blood flowing under the user's skin. The optical sensor can be incorporated as part of a watch or bracelet, for example, that can be worn on the wrist when the user desires to monitor heart rate or during other times as well.

### BRIEF DESCRIPTION OF THE DRAWINGS

In the following the disclosure will be described in greater detail with reference to examples according to the attached drawings, in which

FIG. 1 illustrates an example measuring device in accordance with various aspects of the disclosure;

FIG. 2 is front view of the example device of FIG. 1;

FIG. 3 is a first cross-sectional view of FIG. 2;

FIG. 4 is a second cross-sectional view of FIG. 2;

FIG. 5 is an example watch main unit;

FIG. 6 is an example optical heart rate sensor unit;

FIG. 7 is an example enhanced optical heart rate sensor unit;

FIG. 8 illustrates how light from an LED is reflected from skin to an optical sensor; and

FIG. 9 is an example computing system embodiment.

### DETAILED DESCRIPTION OF EMBODIMENTS

In the following detailed description, numerous specific details are set forth in order to provide a thorough understanding of the present disclosure. However, one skilled in the art will understand that the principles set forth herein may be practiced without these specific details. In other instances, well known methods, procedures, and components have not been described in detail so as not to obscure aspects of the disclosure.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Various embodiments of disclosure are described more fully hereinafter with reference to the accompanying drawings. The disclosed principles may, however, be embodied in many different forms and should not be construed as limited to the embodiments set forth herein. Rather, these embodiments are provided so that this disclosure will be thorough and complete, and will fully convey the scope of the disclosure to those skilled in the art. In the drawings, the size and relative sizes of layers and regions may be exaggerated for clarity.

It will be understood that when an element or layer is referred to as being “on”, “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. It will further be understood that when a particular step of a method is referred to as subsequent to another step, it can directly follow the other step or one or more intermediate steps may be carried out before carrying out the particular step. Like numbers refer to like elements throughout. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the spirit and scope of this disclosure.

Spatially relative terms, such as “beneath”, “below”, “lower”, “above”, “upper” and the like, may be used herein for ease of description to describe one element or feature's relationship to another element(s) or feature(s) as illustrated in the figures.

It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations) and the spatially relative descriptors used herein interpreted accordingly.

Various embodiments are described herein with reference to cross-section illustrations that are schematic illustrations of idealized embodiments (and intermediate structures). As such, variations from the shapes of the illustrations as a result, for example, of manufacturing techniques and/or tolerances, are to be expected. Thus, embodiments should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. All publications, patent applications, patents, and other references mentioned herein are incorporated by reference in their entirety. In case of conflict, the present specification, including definitions, will control. In addition, the materials, methods, and examples are illustrative only and not intended to be limiting.

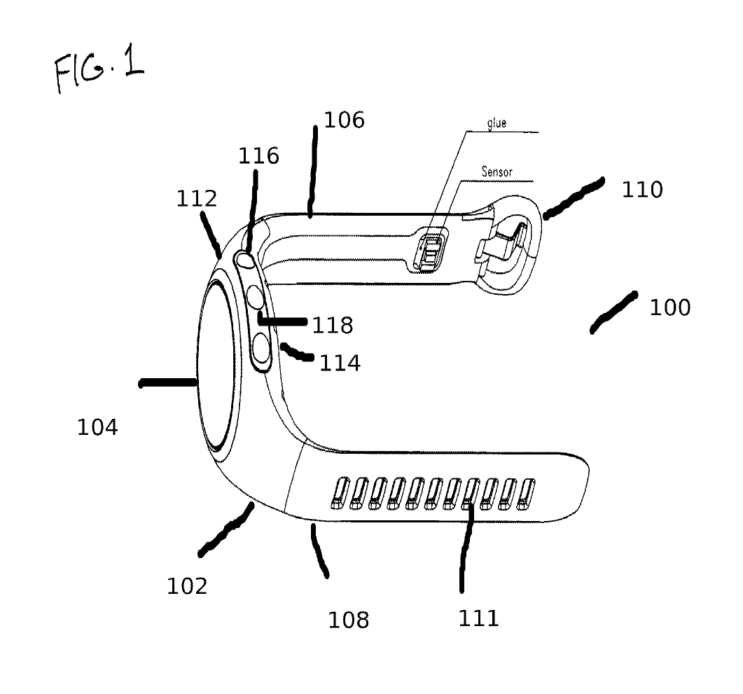

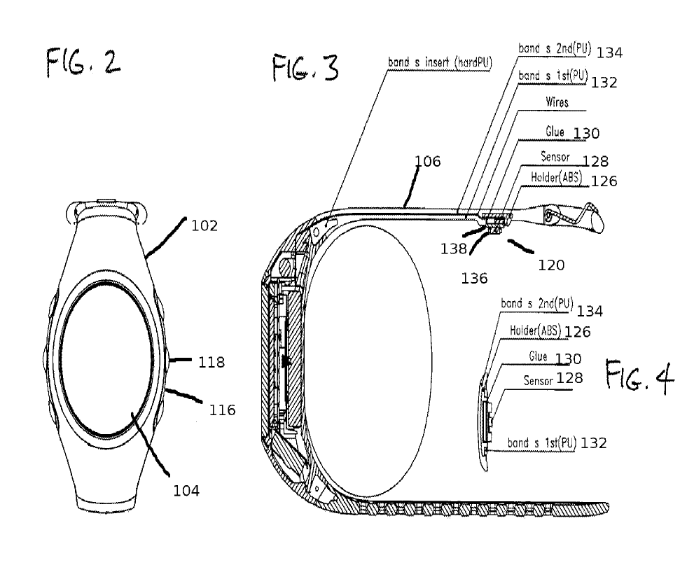

FIG. 1 schematically shows an example measuring device 100, embodied as a watch in this example, in accordance with various aspects of the disclosure. FIG. 2 illustrates a front view of the example measuring device 100. As shown, the measuring device 100 may include a case 102, a display 104, a first strap 106, and a second strap 108. Referring to FIGS. 1 and 2, the display 104 can be coupled with or integrated into the case 102. In one variation, the user can swap, remove, or replace the display 104 or the case 102. The first and second straps 106, 108 are coupled with the case 102 at opposite sides thereof. The first and second straps 106, 108 may also be coupled with one another by a buckle 110 associated with the first strap 106 configured to cooperate with one of the holes 111 in the second strap 108.

In some aspects, the display 104 may be a liquid crystal (LCD) display, a light-emitting diode (LED) display, or the like covered by a protective coating or lens, such as for example an acrylic, glass, or sapphire lens. The case 102 may include a top cover 112 and a case back 114, which may be coupled to one another by any conventional means. The measuring device 100 may include a control module 116 disposed and mounted inside the case 102. The case 102 may also include one or more control buttons 118 associated therewith. The control buttons 118 are configured to be electrically connected with the control module 116 in order to control operation of the measuring device. Alternatively, various other input devices and approaches can be substituted for the control buttons 118 or incorporated with the control buttons 118. These other types of input devices and approaches can include a touchscreen, an external keyboard or pointing device, an infrared remote control, a network-based remote control device, voice input, gesture input, gyroscopic input, and so forth. The control module 116 may include a memory configured to store user information, measurement data, or other information for the user. The control module 116 may be electrically coupled with the display 104 to present a visual representation of data to a user. The control module 116 can communicate information received via the optical heart rate sensor module through a network interface to a remote display or storage device, such as a smartphone, tablet, other wearable computing device, a remote server, a web service, a social network server, and so forth.

Referring now to FIG. 3, the first strap 106 can include an optical heart rate sensor module 120. The placement of the optical heart rate sensor module 120 can be on either strap 106, 108 in the position shown in FIG. 3, or in some other position. The optical heart rate sensor module 120 can also optionally be located on a wrist-facing surface of the case 102. The measuring device 100 can include one or more optical heart rate sensor modules 120. The first strap 106 can further include a first strap member 122 and a second strap member 124 co-molded together over the optical heart rate sensor module 120. The first strap member 122 and the second strap member 124 can be produced separately and bonded together, or can be co-extruded over the sensor module. The optical heart rate sensor module 120 can include a metal shield/holder 126 and an optical sensor unit 128 such as, for example, a printed circuit board including, for example, a controller, an amplifier, and a filter. A metal shield 126 can be coupled to a first side 132 of the optical sensor unit 128 via an adhesive 130 such as, for example, Black Epoxy. The shield 126 can alternatively be composed of a different, non-metal material, such as glass, plastic, or fabric. The shield 126 can be a solid plate, or can have one or more holes to save space and/or weight, such as a plate having a grid of holes. The adhesive 130 may also electrically and/or thermally insulate the optical sensor unit 128 from the metal shield/holder 126. A second side 134 of the optical sensor unit 128, which faces a direction opposite to the first side 124, may include one or more light sources 136 such as, for example, light emitting diodes (LEDs) configured to project light toward the skin of a user. The LEDs can be tuned to project light of a specific frequency or intensity, and can be adjusted by the control module 116 for different skin types, for sensing different aspects of the heart rate of the user, for different energy consumption characteristics, for different desired degrees of accuracy, and so forth. Alternatively, the user can manually control light projection variables of the LEDs, such as frequency or intensity. In one variation, different sets of LEDs having different light projection characteristics can be activated separately or in conjunction with each other to achieve a desired type of light and reflections. The optical heart rate sensor module 120 can include fully or partially transparent shields, covers, or lenses proximate to the LEDs through which light is projected to the skin of the user. Lenses can focus and direct the light from the LEDs for a more precise path intended to reflect to a specific optical sensor or sensors 138. The second side 134 of the optical sensor unit 128 may also include an optical sensor 138 proximal the light source(s) and configured to detect light reflected from the skin of a user.

The optical sensor 138 can be sufficiently sensitive to detect the movement of blood through blood vessels at the wrist of a user by measuring the amount of light absorbed by the skin based on reflections of the light off the skin into the optical sensor 138. The optical sensor 138 is very sensitive to light, electrical static, and electromagnetic waves. Thus, the shield/holder 126 and adhesive 130 form a chamber about the optical sensor unit 128 to protect the sensor unit 128 so that the sensor unit 128 can work when worn on the wrist of a user. The shield/holder 126 and adhesive 130 protect the sensor unit 128 from ambient light, static electricity, and electromagnetic waves. This also helps to detect the weak blood flow signal under the user's skin.

Devices of the present disclosure are suitable for use in all types of measuring devices, which are carried by a user during exercise or other non-exercise activities for non-invasively measuring at least one signal from the body, for example, in heart rate monitors, and even in advanced versions of heart rate monitors in which, for example, the user's energy consumption, blood pressure, etc. are measured in addition to or instead of the heart rate. A sufficiently sensitive sensor may be able to determine, at least partially, other attributes of the blood, such as viscosity, pressure, or chemical and/or fluid make-up of the blood.

In one embodiment of the present disclosure, the measuring device 100 includes a heart rate monitor. The control module 116 of the heart rate monitor may include a measuring unit and a control unit for controlling the measuring unit. The control module 116 also controls a user interface comprising control buttons 118 and display 104. The control unit may be a microprocessor comprising an ROM memory in which the software controlling the device is stored. The control module 116 may further comprise additional memory in which information on, for example, heart rate gathered during the measurement can be stored. In principle, the control unit can also be implemented by an ASIC circuit or by another coupling composed of HW units. Thus the changes according to the disclosure in the measuring device are preferably changes in the software of the device.

The measuring unit may be one piece, for example, a heart rate monitor carried on the wrist. The heart rate may be measured from the wrist. However, in some aspects, a measurement result may be obtained by present technology by using a solution of the type described, in which the measuring unit is divided into two parts: a wireless transmitter that is attached around the chest and measures the heart rate, and a heart rate receiver in the measuring device 100 attached to the wrist.

In various aspects, the display 104 may display in watch mode the time of day, the date, or other watch or calendar data. In an enhanced watch or calendar mode, the display 104 can present weather data, upcoming appointments, and so forth. The display 104 may display a heart symbol for indicating whether heart rate measurement is active. The control buttons 118 shown in the figure constitute the selection means intended for shifting between different modes and displays. For example, one button may be used for making selections and for starting and stopping functions. One button may be intended for adjusting the settings and for using a background light of the display 104 and a sound signal during a measurement function. The measuring device 100 can further communicate wirelessly with other devices, such as a cellular phone, for displaying or communicating heart rate measurement data. The display 104 can render historical heart rate measurements, as well. For example, the display 104 can render a heart rate of the user during a similar exercise in the last week or month, for example. In this way, the user can have a point of reference for comparison with the current heart rate information.

The measuring device 100 may allow the user e.g. to program a short-cut function for a selection means in set mode. This short-cut allows the user to rapidly access a frequently used function. Another feature facilitating the use is a home selection function that allows the user to rapidly access the basic mode of the device, e.g. watch mode. Both the short-cut and home selection can be implemented in various ways, as would be appreciated by persons skilled in the art. An alternative is to simultaneously press several buttons, for example, two different buttons, to make the selection. Another alternative is to press the button for an extended period of time, for example, two seconds, after which the selection is made.

FIG. 5 is an example watch main unit 500. In this example, the cover 506 is attached to a holder 504 via a set of spring contacts 502. The cover 506 can house a PCB 516, an LCD connector 514, a backlight 512, and a display 510 such as an LCD. The holder 504 can further house a switch contact 518 for switches or buttons for user input, as well as a battery 520.

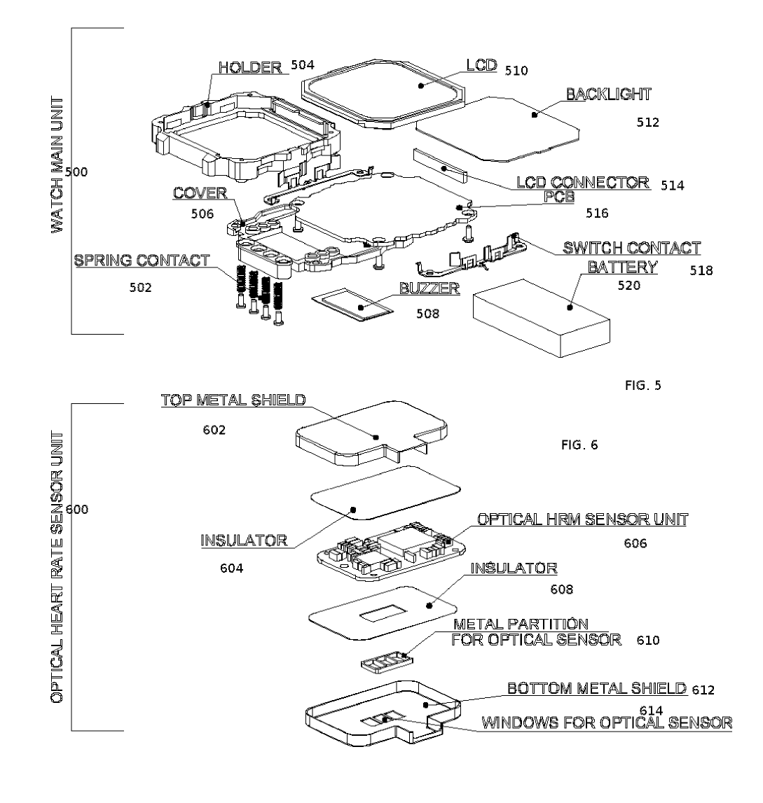

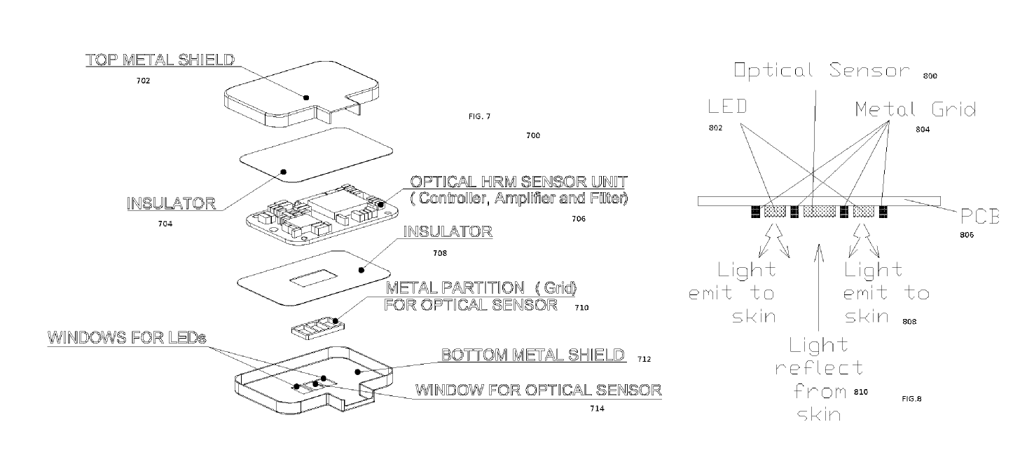

FIG. 6 is an example optical heart rate sensor unit 600. In this example, a top shield 602, which can be made of metal or some other material, can optionally house a first insulator 604, an optical heart rate monitor sensor unit 606, a second insulator 608, and a metal partition 610 for the optical sensor. Then a bottom metal shield 612 can include holes or windows 614 for the optical sensor so light can be emitted onto the skin and reflected back to the optical heart rate monitor sensor unit 606.

FIG. 7 is an example enhanced optical heart rate sensor unit 700. In this example, a top shield 702, which can be made of metal or some other material, can optionally house a first insulator 704, an optical heart rate monitor sensor unit 706, a second insulator 708, and a metal partition 710 or grid for the optical sensor. The optical heart rate monitor sensor unit 706 as shown in this example includes a controller, an amplifier, and a filter. Then a bottom metal shield 712 can include holes or windows 714 for the optical sensor so light can be emitted onto the skin and reflected back to the optical heart rate monitor sensor unit 606. The monitor sensor unit 706 can be mounted on a PCB, which can be protected by the insulator on both top and bottom, and mounted inside the top shield 702 and the bottom shield 712. A metal grid 710 can be mounted around the LEDs and the optical sensor. The metal grid can be used to avoid light interference from two LEDs. The optical only hole in the grid can pick up signal from the reflection from skin. The bottom shield 712 can include three holes to allow light to emit out to skin through the outer holes. The optical sensor 706 at the middle hole can receive light reflected from blood underneath skin.

The back of the watch case can include soft plastic around the optical sensor to prevent ambient light from negatively affecting the accuracy of the blood flow information. Other types of padding or light barriers can be used, such as felt or foam padding, fabric, gel, and so forth.

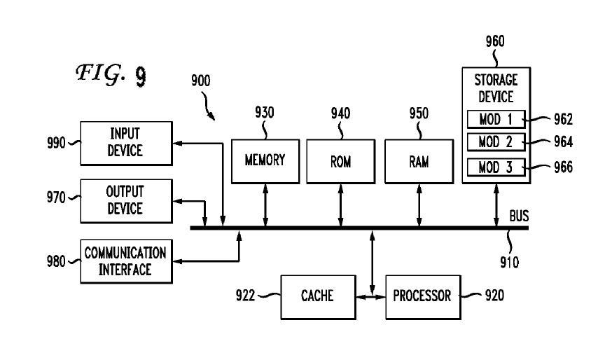

FIG. 8 illustrates how light from an LED 802 is reflected from skin to an optical sensor 800. The LED 802, which can be mounted on a PCB 806, emits light onto the skin 808. A metal grid 804, or a grid of some other material, supports and separates the LEDs 802 from the optical sensor 800. The emitted light 808 is reflected from the skin 810 to an optical sensor 800. The optical sensor 800 can process the reflected light to determine heart rate information based on how much light is absorbed by the skin. Alternatively, the optical sensor 800 can pass received information about the reflected light to a processing module that can determine heart rate information.

The LED 802 can be configured to emit light continuously for a desired duration, and the optical sensor 800 can be configured to sense reflected light continuously for the desired duration. For example, the LED 802 can transmit continuously for a desired monitoring period of 5 minutes, and the optical sensor 800 can sense reflected light for all of those 5 minutes. Alternatively, to conserve power, the LED 802 can be configured to emit light at non-continuous intervals for the desired duration. For example, the LED 802 can be configured to emit light for 10 seconds every minute, and the optical sensor 800 can be configured to sense reflected light for each non-continuous interval. Alternatively, the optical sensor 800 can be configured to receive light continuously, while the LED 802 is configured to transmit only periodically. The behavior of the LED 802 and the optical sensor 800 can be user-configurable, enabling a user to monitor heart rate information on a continuous real-time basis if the exact heart rate each second is important, while also enabling the user to monitor heart rate at some periodic interval to conserve battery power. The measuring device 100 can be configured to automatically enter a power saving mode by emitting light at non-continuous intervals when available battery power is below a threshold, or based on some other power-related event.

FIG. 9 is an example computing system embodiment 900. With reference to FIG. 9, an exemplary system and/or computing device 900 includes a processing unit (CPU or processor) 920 and a system bus 910 that couples various system components including the system memory 930 such as read only memory (ROM) 940 and random access memory (RAM) 950 to the processor 920. The system 900 can include a cache 922 of high-speed memory connected directly with, in close proximity to, or integrated as part of the processor 920. The system 900 copies data from the memory 930 and/or the storage device 960 to the cache 922 for quick access by the processor 920. In this way, the cache provides a performance boost that avoids processor 920 delays while waiting for data. These and other modules can control or be configured to control the processor 920 to perform various operations or actions. Other system memory 930 may be available for use as well. The memory 930 can include multiple different types of memory with different performance characteristics. It can be appreciated that the disclosure may operate on a computing device 900 with more than one processor 920 or on a group or cluster of computing devices networked together to provide greater processing capability. The processor 920 can include any general purpose processor and a hardware module or software module, such as module 1 962, module 2 964, and module 3 966 stored in storage device 960, configured to control the processor 920 as well as a special-purpose processor where software instructions are incorporated into the processor. The processor 920 may be a self-contained computing system, containing multiple cores or processors, a bus, memory controller, cache, etc. A multi-core processor may be symmetric or asymmetric. The processor 920 can include multiple processors, such as a system having multiple, physically separate processors in different sockets, or a system having multiple processor cores on a single physical chip. Similarly, the processor 920 can include multiple distributed processors located in multiple separate computing devices, but working together such as via a communications network. Multiple processors or processor cores can share resources such as memory 930 or the cache 922, or can operate using independent resources. The processor 920 can include one or more of a state machine, an application specific integrated circuit (ASIC), or a programmable gate array (PGA) including a field PGA.

The system bus 910 may be any of several types of bus structures including a memory bus or memory controller, a peripheral bus, and a local bus using any of a variety of bus architectures. A basic input/output (BIOS) stored in ROM 940 or the like, may provide the basic routine that helps to transfer information between elements within the computing device 900, such as during start-up. The computing device 900 further includes storage devices 960 or computer-readable storage media such as a hard disk drive, a magnetic disk drive, an optical disk drive, tape drive, solid-state drive, RAM drive, removable storage devices, a redundant array of inexpensive disks (RAID), hybrid storage device, or the like. The storage device 960 can include software modules 962, 964, 966 for controlling the processor 920. The system 900 can include other hardware or software modules. The storage device 960 is connected to the system bus 910 by a drive interface. The drives and the associated computer-readable storage devices provide non-volatile storage of computer-readable instructions, data structures, program modules and other data for the computing device 900. In one aspect, a hardware module that performs a particular function includes the software component stored in a tangible computer-readable storage device in connection with the necessary hardware components, such as the processor 920, bus 910, display 970, and so forth, to carry out a particular function. In another aspect, the system can use a processor and computer-readable storage device to store instructions which, when executed by the processor, cause the processor to perform operations, a method or other specific actions. The basic components and appropriate variations can be modified depending on the type of device, such as whether the device 900 is a small, handheld computing device, a desktop computer, or a computer server. When the processor 920 executes instructions to perform “operations”, the processor 920 can perform the operations directly and/or facilitate, direct, or cooperate with another device or component to perform the operations.

Although the exemplary embodiment(s) described herein employs the hard disk 960, other types of computer-readable storage devices which can store data that are accessible by a computer, such as magnetic cassettes, flash memory cards, digital versatile disks (DVDs), cartridges, random access memories (RAMs) 950, read only memory (ROM) 940, a cable containing a bit stream and the like, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 900, an input device 990 represents any number of input mechanisms, such as a microphone for speech, a touch-sensitive screen for gesture or graphical input, keyboard, mouse, motion input, speech and so forth. An output device 970 can also be one or more of a number of output mechanisms known to those of skill in the art. In some instances, multimodal systems enable a user to provide multiple types of input to communicate with the computing device 900. The communications interface 980 generally governs and manages the user input and system output. There is no restriction on operating on any particular hardware arrangement and therefore the basic hardware depicted may easily be substituted for improved hardware or firmware arrangements as they are developed.

For clarity of explanation, the illustrative system embodiment is presented as including individual functional blocks including functional blocks labelled as a “processor” or processor 920. The functions these blocks represent may be provided through the use of either shared or dedicated hardware, including, but not limited to, hardware capable of executing software and hardware, such as a processor 920, that is purpose-built to operate as an equivalent to software executing on a general purpose processor. For example the functions of one or more processors presented in FIG. 9 may be provided by a single shared processor or multiple processors. (Use of the term “processor” should not be construed to refer exclusively to hardware capable of executing software.) Illustrative embodiments may include microprocessor and/or digital signal processor (DSP) hardware, read-only memory (ROM) 940 for storing software performing the operations described below, and random access memory (RAM) 950 for storing results. Very large scale integration (VLSI) hardware embodiments, as well as custom VLSI circuitry in combination with a general purpose DSP circuit, may also be provided.

The logical operations of the various embodiments are implemented as: (1) a sequence of computer implemented steps, operations, or procedures running on a programmable circuit within a general use computer, (2) a sequence of computer implemented steps, operations, or procedures running on a specific-use programmable circuit; and/or (3) interconnected machine modules or program engines within the programmable circuits. The system 900 shown in FIG. 9 can practice all or part of the recited methods, can be a part of the recited systems, and/or can operate according to instructions in the recited tangible computer-readable storage devices. Such logical operations can be implemented as modules configured to control the processor 920 to perform particular functions according to the programming of the module. For example, FIG. 9 illustrates three modules Mod1 962, Mod2 964 and Mod3 966 which are modules configured to control the processor 920. These modules may be stored on the storage device 960 and loaded into RAM 950 or memory 930 at runtime or may be stored in other computer-readable memory locations.

One or more parts of the example computing device 900, up to and including the entire computing device 900, can be virtualized. For example, a virtual processor can be a software object that executes according to a particular instruction set, even when a physical processor of the same type as the virtual processor is unavailable. A virtualization layer or a virtual “host” can enable virtualized components of one or more different computing devices or device types by translating virtualized operations to actual operations. Ultimately however, virtualized hardware of every type is implemented or executed by some underlying physical hardware. Thus, a virtualization compute layer can operate on top of a physical compute layer. The virtualization compute layer can include one or more of a virtual machine, an overlay network, a hypervisor, virtual switching, and any other virtualization application.

The processor 920 can include all types of processors disclosed herein, including a virtual processor. However, when referring to a virtual processor, the processor 920 includes the software components associated with executing the virtual processor in a virtualization layer and underlying hardware necessary to execute the virtualization layer. The system 900 can include a physical or virtual processor 920 that receive instructions stored in a computer-readable storage device, which cause the processor 920 to perform certain operations. When referring to a virtual processor 920, the system also includes the underlying physical hardware executing the virtual processor 920.

Embodiments within the scope of the present disclosure may also include tangible and/or non-transitory computer-readable storage devices for carrying or having computer-executable instructions or data structures stored thereon. Such tangible computer-readable storage devices can be any available device that can be accessed by a general purpose or special purpose computer, including the functional design of any special purpose processor as described above. By way of example, and not limitation, such tangible computer-readable devices can include RAM, ROM, EEPROM, CD-ROM or other optical disk storage, magnetic disk storage or other magnetic storage devices, or any other device which can be used to carry or store desired program code in the form of computer-executable instructions, data structures, or processor chip design. When information or instructions are provided via a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

In the claims the word “comprising” does not exclude other elements or steps, and the indefinite article “a” or “an” does not exclude a plurality. A single component or other unit may fulfil the functions of several items recited in the claims. The mere fact that certain measures are recited in mutually different claims does not indicate that a combination of these measures cannot be used to advantage. Any reference signs in the claims should not be construed as limiting the scope.

What is claimed is:

1. A measuring device configured to be worn on a wrist of a user, the measuring device comprising:
a processor;
a display;
a case enclosing the display and the processor;
a first strap coupled to a first side of the case;
a second strap coupled a second side of the case opposite the first side;
an optical heart rate sensor module incorporated into the first strap, wherein the optical heart rate sensor module faces skin of the user while worn by the user, and wherein the optical heart rate sensor module comprises a grid, a light source configured to emit light through the grid at the skin of the user, and an optical sensor configured to receive, through the grid, light emitted by the light source and reflected off the skin, and wherein the processor is configured to calculate heart rate data of the user based on information gathered via the optical sensor.

2. The measuring device of claim 1, wherein the grid includes a first hole for the light source, and a second hole for the optical sensor.

3. The measuring device of claim 1, wherein the case further encloses a control module communicatively connected to the display and to the optical heart rate sensor module.

4. The measuring device of claim 3, further comprising a user input device communicatively connected to the control module.

5. The measuring device of claim 1, further comprising a memory.

6. The measuring device of claim 1, wherein the memory is configured to store user information and measurement data received from the optical heart rate sensor module.

7. The measuring device of claim 1, wherein the optical heart rate sensor module is incorporated into the first strap via a first strap member and a second strap member co-molded together over the optical heart rate sensor module.

8. The measuring device of claim 1, wherein the optical heart rate sensor module is incorporated into the first strap via a first strap member and a second strap member that are bonded together around the optical heart rate sensor module.

9. The measuring device of claim 1, wherein optical heart rate sensor module further comprises a shield housing the grid, the light source, and the optical sensor.

10. The measuring device of claim 9, wherein a skin-facing surface of the shield includes a set of windows corresponding to holes in the grid.

11. The measuring device of claim 9, wherein the optical sensor further comprises a controller, an amplifier, and a filter.

12. The measuring device of claim 9, wherein the controller, the amplifier, and the filter are incorporated onto a single circuit board.

13. The measuring device of claim 12, wherein the shield further houses a first insulator layer between the single circuit board and a top inner surface of the shield, and a second insulator layer between the single circuit board and a bottom inner surface of the shield.

13. The measuring device of claim 1, wherein the processor is configured to render at least part of information received via the optical heart rate sensor module on the display.

14. The measuring device of claim 1, the measuring device further comprising a power source and a speaker.

15. The measuring device of claim 1, wherein the display is configured to render at least one of calendar data or time data and a visual indicator of whether the optical heart rate sensor module is active.

16. The measuring device of claim 1, wherein the light source is configured to emit light at an indicated intensity and at an indicated frequency.

17. The measuring device of claim 16, wherein at least one of the indicated intensity or the indicated frequency is user-selectable.

18. The measuring device of claim 1, wherein the light source is configured to emit light continuously for a desired duration, and the optical heart rate sensor module is configured to sense reflected light continuously for the desired duration.

19. The measuring device of claim 1, wherein the light source is configured to emit light at non-continuous intervals, and the optical heart rate sensor module is configured to sense reflected light for each non-continuous interval.

20. The measuring device of claim 1, further comprising a network interface for communicating at least part of information received via the optical heart rate sensor module to a remote display device.
